(19) United States
(12) Patent Application Publication
(10) Pub. No.: US 2019/0111250 A1
(43) Pub. Date: Apr. 18, 2019

(54) ELECTRODE ASSEMBLY FOR METAL-SENSITIVE NEUROLOGICAL MONITORING

(71) Applicant: RHYTHMLINK INTERNATIONAL, LLC., COLUMBIA, SC (US)

(72) Inventors: SHAWN V. REGAN, COLUMBIA, SC (US); DANIEL MCCOY, MURRELLS INLET, SC (US); HARRISON M. FLOYD, COLUMBIA, SC (US); LEAH HANSON, NEW GLARUS, WI (US); MICHAEL J. O'LEARY, COLUMBIA, SC (US)

(73) Assignee: RHYTHMLINK INTERNATIONAL, LLC., COLUMBIA, SC (US)

(21) Appl. No.: 16/067,467

(22) PCT Filed: Jul. 13, 2017
(86) PCT No.: PCT/US17/41834

§ 371 Date: Jun. 29, 2018

Related U.S. Application Data
(60) Provisional application No. 62/361,818, filed on Jul. 13, 2016.

(51) Int. Cl. A61N 1/08; G01R 33/28; A61N 1/05; A61B 5/055

(52) U.S. Cl. CPC A61N 1/086; A61B 5/055; A61N 1/0526; G01R 33/285

(57) ABSTRACT

An electrode assembly for use in computer tomographic (CT) scans and magnetic resonance imaging (MRI) procedures include lead wires of 40 gauge copper electrical conductors, free of tin, in PVC insulation attached to electrodes. The electrodes, which may also be made of silver, gold or silver chloride or be made of metal-filled or metal-coated plastic, are attached to lead wires by any of the following (1) crimping using copper or polymer crimps; (2) shrink-wrap connections or ties, (3) radio frequency welding or heat staking; or (4) using conductive adhesives. The present electrode assembly reduces artifacts in the resulting images during CT scans and MRI procedures and thereby results in clearer images.

### TECHNOLOGICAL FIELD

This disclosure relates generally to the field of electrodes for neurological monitoring, and more specifically to electrodes for use during computerized tomography scanning and magnetic resonance imaging.

### BACKGROUND

Neurological monitoring electrodes are used to obtain signals from the brain. Electrodes may be attached to the scalp of a patent by using adhesives or by insertion directly into the surface of the scalp. The electrodes are connected by wire conductors to a device that receives electrical signals, processes them and displays the processed signals for technicians and physicians to evaluate. The signals may be processed, for example, to provide two-dimensional images of the brain so that the sources of signals and their relative positions can be readily located.

Certain types of procedures, notably computerized tomography (CT) scans and magnetic resonance imaging (MRI) can detect metal and indeed react with it. CT scans are essentially a series of X-rays in the wavelength range of 10-0.01 nm. MRI's operate based on a rapidly changing magnetic field in the radio wave wavelength region of the electromagnetic spectrum, with a wavelength of about a meter. Copper is generally not magnetic; tin is not magnetic. Tin is less transparent to X-rays than copper.

To avoid artifacts in CT and MRI images, monitoring electrodes, which are made of tinned copper, may be temporarily removed during CT and MRI procedures. If electrodes or other metal objects are left in place, the presence of metal results in artifacts appearing in the CT and MRI images. Artifacts may interfere with the evaluation of the images and certainly degrade the quality of the images. FIG. 2 illustrates a CT scan that is an extreme example of artifacts in an image, namely, radial streaks that obscure much of the useful detail of this CT scan.

While removing the electrodes before CT and MRI procedures and reinstalling them afterwards involves additional time, expense and risk of mistakes, it can reduce the number of artifacts and thereby preserve the image quality.

A way to reduce artifacts without removal of the electrodes and their lead wires before beginning CT scans and MRI procedures would be an advantage.

### SUMMARY OF THE DISCLOSURE

Disclosed herein is an electrode assembly that provides significant reduction in artifacts when the electrode assembly remains attached to the scalp of a patient during CT scans and MRI. The reduction results from material selection and connection technique, the use of multiple, thinner wires in the conductor, and the use of copper electrodes that without tin.

An aspect of the disclosure is an electrode assembly including an electrical lead wire having a first end and a second end. The lead wire includes at least one conductor made of copper without a tin coating, save perhaps at its ends and an electrode attached to the second end of the lead wire. The electrical conductors in a lead wire are at least 39 gauge and may be 40 gauge. There may be as many as 19 conductors in lead wire.

Another aspect of the disclosure is that the lead wire may be welded or heat-staked to the electrode.

Another aspect of the disclosure is that the lead wire and electrode may be attached together using a copper crimp or a polymeric crimp.

An aspect of the disclosure is that the lead wire and the electrode may be attached together using heat-shrinkable tubing, which may be conductive shrinkable tubing, or a plastic tie.

Those skilled in neurological monitoring will appreciate these and other features of the disclosure from a careful reading of the Detailed Description accompanied by the drawings.

### BRIEF DESCRIPTION OF THE DRAWINGS

FIGS. 1A and 1B illustrate in perspective an electrode assembly for neurological monitoring, including its electrode, lead wire, and plug, and, in a perspective, cross-sectional view, the interior of a portion of the lead wire, respectively;

FIG. 2 is an extreme example of the effect of an artifact in a CT scan of a human skull;

FIG. 3 is in image made during a CT scan of a first image phantom using prior art electrodes;

FIG. 4 is an image made during of a CT scan of the same image phantom as used in FIG. 3 and with the present electrodes replacing the prior art electrodes;

FIG. 5 is an image made during a CT scan of a second image phantom using prior art electrodes;

FIG. 6 is an image made during a CT scan of the same image phantom as used in FIG. 5 and with the present electrodes replacing the prior art electrodes;

FIG. 7 is an image made during a CT scan with the present electrodes of a first plane of a patient's head showing artifacts, according to an aspect of the disclosure; and

FIG. 8 is an image during a CT scan of a different plane of the head of the patient than that shown in FIG. 7 with the present electrode attached, according to an aspect of the disclosure.

### DETAILED DESCRIPTION

In the description of the present electrode assembly, the term “electrode assembly” refers to three components: the electrode, the lead wire that is connected to and runs from the electrode, and the plug on the other end of the lead wire, and also includes the connections between these three components.

The term lead wire refers to an insulated electrical conductor or bundle of electrical conductors. A bundle of electrical conductors may also be referred to herein as a plurality of electrical conductors.

Copper wire is generally tinned, covered with a layer of tin giving it a silver appearance rather than a copper appearance to make it easier to solder. The tin also helps to prevent corrosion. Other coatings may be used in place of tin, such as silver and gold.

The improvement in prior art electrode assemblies disclosed and described herein makes them suitable for leaving in place during a CT or MRI procedure, and thereby avoids having to remove them for the procedure and then reattach them afterwards. The removal and reattachment take time, which is expensive in the context of medical procedures, and also increased the possibility of errors or mistakes. The improvement directly results in fewer, smaller, less intrusive artifacts being visible in the images made by CT scans and MRI procedures. It is because of the artifacts left by prior art electrode assemblies during these procedures that the prior art electrodes are best removed prior to the commencement of these procedures.

In order to improve prior art electrodes and make them suitable for remaining attached to a patient's head during a CT scan or MRI procedure and reduce the impact of artifacts in the resulting images, a number of improvements are described herein to the basic materials and their connections over the prior art electrode assembly. The prior art electrode assembly may include an electrode soldered to a lead wire with a plug on the distal end of the lead wire. The plug is formed to insert into a neurological monitoring device and thereby be held there and have the electrode be in electrical connection with the device.

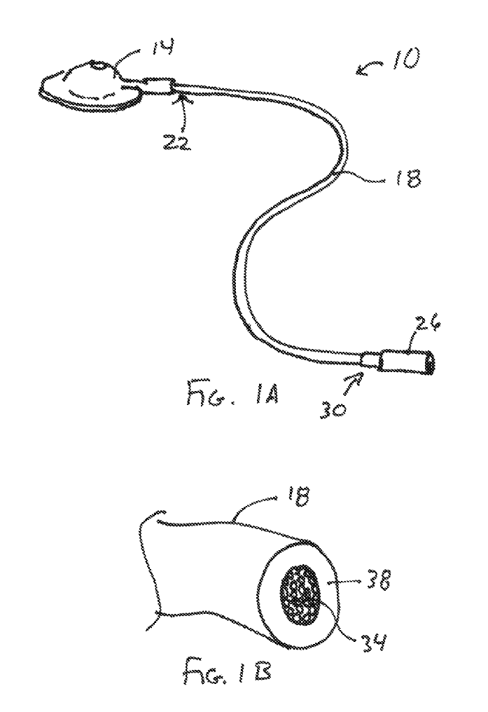

An improved electrode assembly, generally indicated by reference number 10, in FIGS. 1A and 1B, is physically very similar externally and internally to a prior art electrode assembly, and is used in the same way. However, the improved electrode assembly 10 is less visible to the wavelengths used in CT scans and MRI procedures and therefore produce significantly fewer artifacts.

As shown in FIGS. 1A and 1B, electrode assembly 10, includes an electrode 14 and a lead wire 18 with a first end 22 attached to electrode 14, and a second end 30 attached to a plug 26. Inside lead wire 18 are electrical conductors 34, best seen in FIG. 1B, surrounded by insulation 38. Insulation 38 may be medical grade, polyvinyl chloride (PVC).

Electrical conductors 34 in the present electrode assembly 10 are thinner than conductors in prior art electrode assemblies. The number of individual electrical conductors 34 may be at least the same as that used in the prior art. For example, prior art lead wires comprise 19 electrical conductors of 38-gauge tinned copper wire insulated from the surroundings by medical grade PVC. Lead wire 18 may also include 19 electrical conductors, or more, and are made of 40-gauge untinned copper wire insulated in medical grade PVC. The finer gauge results in a thinner diameter of electrical conductors 34, namely, 1 mm versus 1.18 mm. The absence of tin results in electrical conductors 34 having a copper-colored appearance rather than silver-colored copper electrical conductors. More importantly, the absence of tin reduces the operating temperature of electrical conductors 34 during CT and MRI procedures and the visibility of the wire to the wavelengths of electromagnetic radiation used in CT and MRI procedures. Thus, use of tin-free copper wire reduces the impact of artifacts and the temperature rise of the electrode assemblies during CT scans and MRI procedures.

Furthermore, in place of a brass electrode as done in prior art electrode assemblies, a silver-filled or silver-coated conductive epoxy electrode or a pure copper electrode may be used. Alternatively, in place of a brass crimp to hold electrode 14 to lead wire 18, a conductive epoxy or heat shrinkable polymeric tube may be used. Alternatively, electrical conductors 34 may be welded to electrode 14 using radio frequency welding or by heat staking, both of which are known means for attaching two members together. In radio frequency welding, a high frequency is used to cause two part to bond together. In heat staking, two or more parts are physically joined in which one of the parts is made of plastic. The plastic part deforms when heat is applied to an adjacent part and wraps around the part to which it is to be attached.

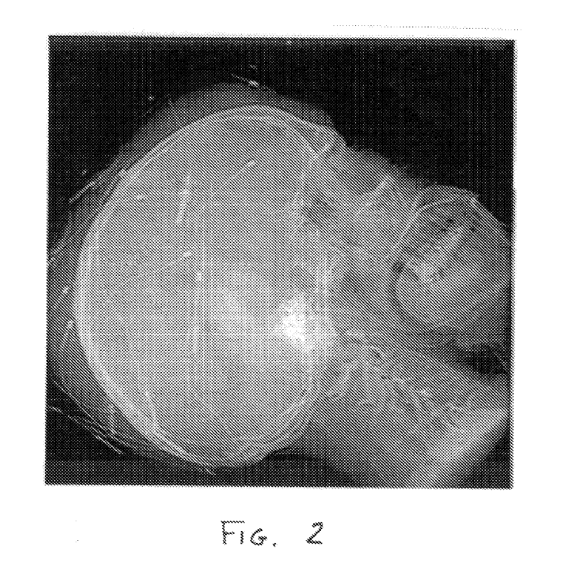

FIGS. 3 and 4 show the reduction in the number and effect of artifacts as a result of replacement of prior art electrode assemblies with the present electrode assemblies 10. The prior art electrode assemblies have lead wires containing 19 strands of 38-gauge, tinned copper wire wrapped in PVC insulation. FIG. 3 shows the result of the CT scan of the prior art electrode assembly. Electrode assemblies 10 may have lead wires 18 containing 19 strands of 40-gauge, untinned copper wire wrapped in PVC insulation. FIG. 4 shows the result of the CT scan on the present electrode assembly 10. FIGS. 3 and 4 both contain phantom images, which are made using a replica of a human head with physical electrodes attached. The replica is then subjected, in this example, to a CT scan. In both FIG. 3 and FIG. 4, the phantom is a profile of a human head next to a vertical line. Both the profile line and the vertical line are used for perspective and to make certain the equipment is operating correctly. The short stray white lines and white specks are artifacts, which are reduced in FIG. 4 compared to FIG. 3, because of the absence of the tin coating on the copper electrical conductors 34. A comparison of FIGS. 3 and 4 shows a significant reduction in artifacts.

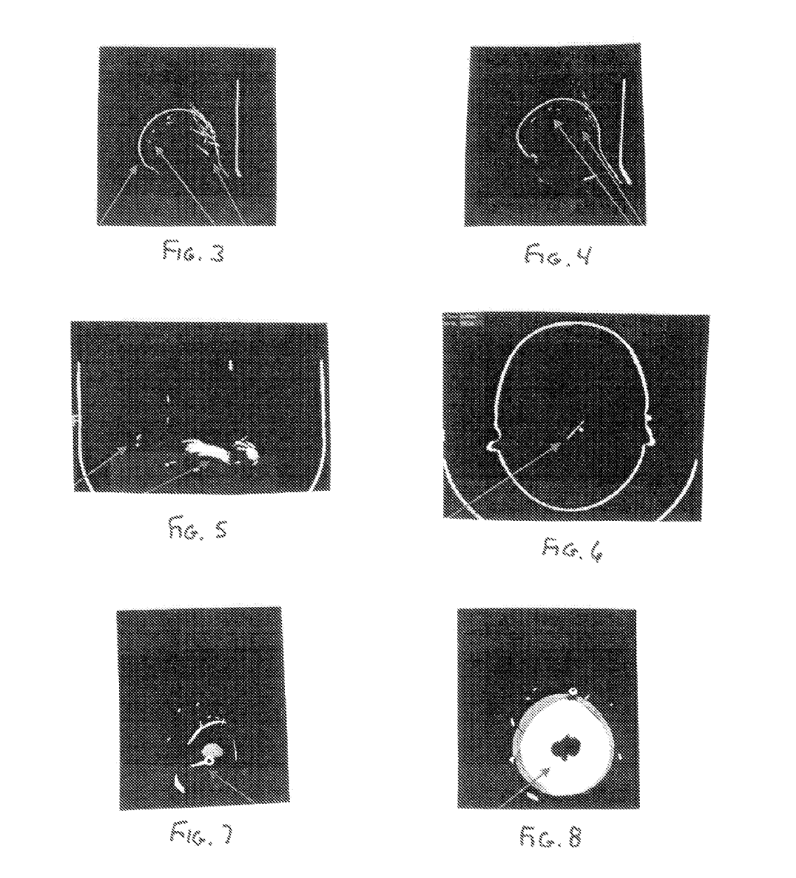

FIGS. 5 and 6 provide a second comparison based on a CT scan of two more phantom images. The heavy white lines are again phantoms used for perspective in testing and are not artifacts of the electrode assemblies. The 19 strands of 38-gauge tinned copper wire wrapped in PVC insulation of the prior art electrode assemblies are used in FIG. 5, These electrode assemblies are replaced by lead wires 18 having 19 strands of 40-gauge untinned copper lead wires 22 wrapped in PVC insulation. The results are shown in FIG. 6. This example also shows a significant reduction in the artifacts.

FIGS. 7 and 8 show a third comparison. These figures show the results of CT scans with an actual human head viewed from the top. Both are images made during CT scans with the present electrode assembly 10, that is, with improved, thinner gauge, untinned copper wire. Both images are made with silver-filled, electrically-conductive epoxy in place of of brass crimps. The two figures are different because they are scans taken at two different planes of the skull. In FIG. 7, the very top of the skull is seen, including the electrode on the vertex itself which appears as a “key” shape in the image. In FIG. 8, the plane of the CT scan is slightly below the top of the skull, so that much of the image includes the bony skull itself, which appears as a white area with a dark center. Significantly, the top or vertex electrode of FIG. 7 is no longer visible.

### Example

A prior art electrode assembly having electrically conducting lead wire (24 cm) with MR PRESSON electrode, sold by Persyst Development Corporation, was exposed in a CT scan. The observed temperature increase of the electrode was 1.6° C. (0.5° C. background) by 1.5 T and 4.9° C. by 3.0 T. A sample of the present electrically conducting wire (24 cm) with the same MR PRESSON electrode attached was also exposed in a CT scan. The observed temperature increase of the electrode was 2.1° C. at 1.5 T and a 2.2° C. at 3.0 T was observed. The difference between the two electrode assemblies is thinner gauge wire (39 gauge versus 38 gauge) and the absence of tin coating in the second electrode assembly. Although the difference in the gauge of the wire and the absence of tin increased the temperature initially, by 0.6° C. at 1.5 T over the prior art electrode assembly, however, thereafter the temperature did not continue to increase appreciably and was well below the 4.9° C. temperature at 3.0 T of the prior art conducting wire. This result for temperature corroborates the results of CT scan images shown in FIGS. 3-6 made because the influence of the CT irradiation spectrum on temperature of the conducting wire also influences visibility in a CT scan of the same conducting wire

Additional measures also reduce artifacts in addition to electrical conductor gauge and the absence of tin. These additional measures include eliminating the brass crimp that holds the electrical conductor to the electrode by using radio frequency welding or heat staking to connect the electrode with the electrical conductors of the lead wire directly. Also, a pure copper electrode or polymeric-based, silver-filled electrode in place of a brass electrode reduces the effect of artifacts on images made during CT and MRI procedures. Alternatively, a different type of mechanical connection may be used in place of a brass crimp, such as use of heat shrinkable tubing to connect lead wire to electrode, or by tying the electrode head to the lead wire electrical conductors.

Minimally, the electrical conductors of the lead wire are tin-free save for their ends and then using tin for effecting a soldered joint. If the gauge of the electrical conductors is increased beyond 39 gauge to 40 gauge, so that the electrical conductors are thinner, the effect on artifacts and temperature during CT or MRI procedures is further reduced. If, the connection between the lead wire and the electrode is not a soldered connection and based on use of electrically conductive, silver-filed epoxy, the improvement in the reduction of artifacts is greater still.

Those skilled in the art of electrodes used in neurological monitoring will appreciate that many substitutions and modifications may be made to the foregoing description of aspects of the disclosed electrode assembly without departing from the spirit and scope of the disclosure.

What is claimed is:

1. An electrode assembly, comprising:
a plug;
a lead wire having a first end and a second end, said first end of said lead wire attached to said plug. said lead wire including at least one electrical conductor made of copper without a tin coating; and
an electrode attached to said second end of said lead wire.

2. The electrode assembly of claim 1, wherein said at least one electrical conductor is at least 39 gauge.

3. The electrode assembly of claim 1, wherein said at least one electrical conductor is at least 40 gauge.

4. The electrode assembly of claim 1, said at least one electrical conductor is at least 19 conductors.

5. The electrode assembly of claim 1, wherein said at least one electrical conductor is radio frequency welded to said electrode.

6. The electrode assembly of claim 1, wherein said at least one electrical conductor is heat staked to said electrode.

7. The electrode assembly of claim 1, further comprising a copper crimp, said copper crimp holding said at least one electrical conductor in electrical connection to said electrode.

8. The electrode assembly of claim 1, further comprising a polymeric crimp, said polymeric crimp holding said at least one electrical conductor in electrical connection to said electrode.

9. The electrode assembly of claim 1, further comprising shrinkable tubing, said shrinkable tubing holding said at least one electrical conductor in electrical connection to said electrode.

10. The electrode assembly of claim 1, further comprising conductive shrinkable tubing, said conductive shrinkable tubing holding said at least one electrical conductor in electrical connection to said electrode.

11. The electrode assembly of claim 1, further comprising a tie holding said at least one electrical conductor in electrical connection to said electrode.

12. The electrode assembly of claim 1, wherein said electrode is made of copper without tin.

13. The electrode assembly of claim 1, further comprising a layer of tin applied to said second end of said at least one electrical conductor wherein said electrode is soldered to said second end.

14. The electrode assembly of claim 1, wherein said electrode is attached to said electrical conductor with an electrically conducting epoxy polymer.

15. The electrode assembly of claim 1, wherein said electrode is a silver-coated epoxy electrode.

16. The electrode assembly of claim 1, wherein said electrode is attached to said electrical conductor using electrically-conducting epoxy.
